(19) United States
(12) Patent Application Publication
(10) Pub. No.: US 2024/0132953 A1
(43) Pub. Date: Apr. 25, 2024

(54) METHODS AND COMPOSITIONS FOR TRACKING BARCODES IN PARTITIONS

(71) Applicant: Bio-Rad Laboratories, Inc., Hercules, CA (US)

(72) Inventors: Man CHENG, Danville, CA (US); Ronald LEBOFSKY, Berkeley, CA (US)

(21) Appl. No.: 18/381,400

(22) Filed: Oct. 17, 2023

Related U.S. Application Data
(60) Provisional application No. 63/417,897, filed on Oct. 20, 2022.

(51) Int. Cl. C12Q 1/6874; C12Q 1/6876

(52) U.S. Cl. CPC C12Q 1/6874; C12Q 1/6876; C12Q 1/682

(57) ABSTRACT

Methods and compositions for generating sequencing reads by partition of origin. One can introduce a unique molecular identifier and bead-specific barcodes to target nucleic acid fragments and the combination of bead-specific barcode, UMI and fragment can be used to identify when multiple bead-specific barcodes originated in the same partition, allowing for improved deconvolution of partition-based sequence analysis.

### CROSS-REFERENCE TO RELATED PATENT APPLICATIONS

The present patent application claims benefit of priority to U.S. Provisional Patent Application No. 63/417,897, filed Oct. 20, 2022, the entirety of which is hereby incorporated by reference for all purposes.

### BACKGROUND OF THE INVENTION

Tagging biological substrates with molecular barcodes in partitions can provide novel biological insight of the substrates that co-localize to discrete partitions, through the sequencing of the molecular barcodes and analysis, thereof. Increasing the number of barcoding competent partitions, such as droplets, increases the number of sequencing based data points and converts a greater fraction of input substrates into data. Barcodes can be delivered to partitions, such as droplets, using beads as the delivery vehicle. Thus, barcode bead overloading in partitions, which results in partitions with more than one bead and increases the percentage of barcoding competent partitions, provides higher substrate to sequencing data conversion rates. However, when two or more barcodes occur in discrete partitions, the substrates and data are split between the two barcodes, creating fractionated data points. The instant disclosure provides a solution to the problems created when more than one barcoded bead is present in a partition, such as problems associated with fractionated data points.

### BRIEF SUMMARY OF THE INVENTION

In some embodiments, methods of sorting sequencing reads by partition of origin are provided. In some embodiments, the method comprises,
providing RNA/cDNA or DNA/cDNA hybrid molecules in fixed and permeabilized cells, wherein the fixed and permeabilized cells comprise cross-linked molecules;
generating random breaks in the hybrid molecules and randomly inserting at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence and wherein the first adaptor oligonucleotide, the second adaptor oligonucleotide, or both further comprise a unique molecular identifier (UMI) sequence;
partitioning the cells into partitions with one or more bead, wherein each bead is linked to multiple copies of a bead-specific barcoding oligonucleotide having an identical 3′ end comprising either the first universal sequence or the second universal sequence, wherein bead-specific barcoding oligonucleotides linked to different beads can be identified by a unique bead-specific barcode in the bead-specific barcoding oligonucleotide and wherein at least some partitions contain at least two different of the beads;
optionally, in the partitions, reversing at least some of the cross-linking in the cross-linked molecules in the cells and/or lysing the cells;
before, during or after the reversing, extending with a polymerase (i) the first 3′ end using the second adaptor oligonucleotide as a template such that the first 3′ end is linked to a reverse complement of the second universal sequence and (ii) the second 3′ end using the first adaptor oligonucleotide as a template such that the second 3′ end is linked to a reverse complement of the first universal sequence, thereby forming gap-filled hybrid molecule fragments;
amplifying in the partitions the gap-filled hybrid molecule fragments by annealing and extending the bead-specific barcoding oligonucleotide to the reverse complements of the first universal sequence or the reverse complements of the second universal sequence on the cDNA fragments to generate amplicons comprising: the bead-specific barcoding oligonucleotide, cDNA fragments, the second universal end sequence and the UMI sequence, under conditions in which if a first bead and a second bead are present in a partition, bead-specific barcoding oligonucleotides from the first and second beads each separately are extended using the same cDNA hybrid molecule fragment as a template to form (i) an amplicon comprising a first bead-specific barcode and a first UMI sequence and (ii) an amplicon comprising a second bead-specific barcode and the first UMI sequence;
nucleotide sequencing amplicons from the amplifying to generate sequencing reads; and sorting sequencing reads from different partitions wherein (i) the amplicon comprising the first bead-specific barcode and the first UMI sequence and (ii) the amplicon comprising the second bead-specific barcode and the first UMI sequence, and (iii) optionally a same fragment break point, are from the same partition.

In some embodiments, the hybrid molecules are RNA/cDNA hybrid molecules and the cDNA is a first strand cDNA. In some embodiments, the RNA/first strand cDNA hybrid molecules are formed by reverse transcribing RNA from the cell with a polyA, random or gene-specific reverse transcription primer. In some embodiments, the hybrid molecules are DNA/cDNA hybrid molecules. In some embodiments, the DNA/first strand cDNA hybrid molecules are formed by polymerase chain reaction or primer extension.

In some embodiments, the first adaptor oligonucleotide comprises a UMI sequence. In some embodiments, the second adaptor oligonucleotide comprises a UMI sequence. In some embodiments, the first adaptor oligonucleotide and the second adaptor oligonucleotide comprises a UMI sequence.

In some embodiments, the first adaptor oligonucleotide, the second adaptor oligonucleotide, or both further comprises a sample barcode sequence.

In some embodiments, the generating comprises contacting the RNA/cDNA hybrid molecules with a transposase that introduces the adaptor oligonucleotides into the RNA/cDNA hybrid molecules.

In some embodiments, the bead-specific barcoding oligonucleotide comprises a 3′ end comprising the first universal sequence and the amplifying comprises annealing and extending the bead-specific barcoding oligonucleotide to the reverse complements of the first universal sequence. In some embodiments, the amplifying further comprises extending a reverse primer having a 3′ end comprising a reverse complement of the second universal sequence using the amplicons as templates.

In some embodiments, the bead-specific barcoding oligonucleotide comprises a 3′ end comprising the second universal sequence and the amplifying comprises annealing and extending the bead-specific barcoding oligonucleotide to the reverse complements of the second universal sequence. In some embodiments, the amplifying further comprises extending a reverse primer having a 3′ end comprising the first universal sequence using the amplicons as templates.

In some embodiments, the partitions are microwells or droplets in an emulsion.

In some embodiments, the cells are mammalian cells. In some embodiments, the cells as prokaryotic cells. In some embodiments, the cells are eukaryotic cells.

In some embodiments, a plurality of partitions, for example as described herein, is provided. In some embodiments, at least some partitions comprise,
fixed and permeabilized cells comprising cross-linked molecules and containing
gap-filled hybrid molecule fragments formed by:
generating random breaks in RNA/cDNA or DNA/cDNA hybrid molecules and randomly inserting at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence and wherein the first adaptor oligonucleotide, the second adaptor oligonucleotide, or both further comprise a unique molecular identifier (UMI) sequence;
partitioning the cells into partitions with one or more bead, wherein each bead is linked to multiple copies of a bead-specific barcoding oligonucleotide having an identical 3′ end comprising either the first universal sequence or the second universal sequence, wherein bead-specific barcoding oligonucleotides linked to different beads can be identified by a unique bead-specific barcode in the bead-specific barcoding oligonucleotide and wherein at least some partitions contain at least two different of the beads; and
extending with a polymerase (i) the first 3′ end using the second adaptor oligonucleotide as a template such that the first 3′ end comprises a reverse complement of the second universal sequence and (ii) the second 3′ end using the first adaptor oligonucleotide as a template such that the second 3′ end comprises a reverse complement of the first universal sequence, thereby forming gap-filled hybrid molecule fragments;
wherein the at least some partitions further comprise the one or more bead.

In some embodiments, the partitions are droplets in an emulsion or microwells.

In some embodiments, the cells are mammalian cells. In some embodiments, the cells are prokaryotic cells. In some embodiments, the cells are eukaryotic cells.

In some embodiments, the first adaptor oligonucleotide comprises a UMI sequence. In some embodiments, the second adaptor oligonucleotide comprises a UMI sequence. In some embodiments, the first adaptor oligonucleotide and the second adaptor oligonucleotide comprises a UMI sequence.

In some embodiments, the first adaptor oligonucleotide, the second adaptor oligonucleotide, or both further comprises a sample barcode sequence.

### BRIEF DESCRIPTION OF THE DRAWINGS

FIG. 1 shows exemplary steps in the methods described herein. In item 1, cells are fixed and permeabilized in bulk (i.e., not in partitions). In item 2, polyA and non-polyA RNAs are reverse transcribed in situ within the permeabilized cells in bulk. In item 3, reverse-transcribed RNA:DNA hybrid molecules are tagmented (i.e., a transposase introduces breaks and inserts adaptor oligonucleotides at the breaks) by UMI and Sample-index labelled transposases. UMI is used in bead-merging; Sample-index is used in combinatorial indexing for increasing cell-throughput. In item 4, cross-linking of tagmented cells is reversed prior to or after being partitioned into a bead-overloaded droplet.

FIG. 2 shows a continuation of exemplary steps from FIG. 1. In item 4, cross-linking of tagmented cells is reversed prior to or after being partitioned into a bead-overloaded droplet (same as last step of FIG. 1). In item 5, after an in-droplet cell-lysis step, gap-filling of the tagmented RNA:DNA hybrids generate cDNA fragments with adapter sequence on both ends. In item 6, the (transcriptome/gene-specific) fragments of a droplet partition are labelled with a unique set of UMIs, which is later tagged with unique cell barcodes (CBCs) within the same partition. In item 7, after a droplet PCR process, fragments associated with a unique set of droplet-specific UMIs are linked to a unique set of cell barcodes within the same droplet.

FIG. 3 shows a continuation of exemplary steps from FIG. 2. In item 8, bead-merging is carried on by using a Jaccard Similarity calculation between library fragments consisting of UMIs and barcode. Barcode pools associated with the same UMI pool are merged and interpretated as a unique partition, therefore from a single-cell.

FIG. 4 shows an aspect of the methods described herein where the starting nucleic acid is RNA/cDNA hybrids, which have been treated with tagmentation transposase to fragment the hybrids and to introduce adaptor oligonucleotides to the resulting fragments.

FIG. 5 shows exemplary adaptor sequences.

FIG. 6 depicts a flow chart as detailed in Example 1.

FIG. 7 shows a ranking plot of arranging the detected partitions (after barcode-merging) with descending order of read counts as explained in Example 2.

FIG. 8 shows a plot of the total number of unique reads per detected partitions that have various number of detected barcodes as described in Example 2.

### DEFINITIONS

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

The term “amplification reaction mixture” refers to an aqueous solution comprising the various reagents used to amplify a target nucleic acid. These include enzymes, aqueous buffers, salts, amplification primers, target nucleic acid, and nucleoside triphosphates. Amplification reaction mixtures may also further include stabilizers and other additives to optimize efficiency and specificity. Depending upon the context, the mixture can be either a complete or incomplete amplification reaction mixture

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

“Primer extension” refers to any method in which a primer is extended in a template-specific manner. Examples of primer extension include, for example, methods in which a primer hybridizes to a template nucleic acid and a polymerase extends the primer in a template-specific manner. In some embodiments, the template is DNA and the polymerase is a DNA polymerase. In some embodiments, the template is RNA and the polymerase is a reverse-transcriptase. Primer extension can also include, for example, template switching (see, e.g., Zhu Y Y, Machleder E M, et al. (2001) Biotechniques, 30(4):892-897; Ramskold D, Luo S, et al. (2012) Nat Biotechnol, 30(8):777-78, and nick polymerization (also referred to as nick translation), the latter involving nicking one strand of a nucleic acid duplex and using the nicked strand as a primer that is extended using the other strand as a template (see, e.g., Leonard G. Davis Ph.D., et al, in Basic Methods in Molecular Biology, 1986).

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, flanked by or a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein a “barcode” is a short nucleotide sequence (e.g., at least about 4, 6, 8, 10, or 12, nucleotides long) that identifies a molecule to which it is conjugated. Barcodes can be used, e.g., to identify molecules in a partition. Such a partition-specific barcode should be unique for that partition as compared to barcodes present in other partitions. For example, partitions containing target RNA from single-cells can subject to reverse transcription conditions using primers that contain a different partition-specific barcode sequence in each partition, thus incorporating a copy of a unique “cellular barcode” into the reverse transcribed nucleic acids of each partition. Thus, nucleic acid from each cell can be distinguished from nucleic acid of other cells due to the unique “cellular barcode.” In some cases, the cellular barcode is provided by a “bead barcode” that is present on oligonucleotides conjugated to a bead, wherein the bead barcode is shared by (e.g., identical or substantially identical amongst) all, or substantially all, of the oligonucleotides conjugated to that bead. Thus, cellular and bead barcodes can be present in a partition, attached to a bead, or bound to cellular nucleic acid as multiple copies of the same barcode sequence. Cellular or bead barcodes of the same sequence can be identified as deriving from the same cell, partition, or bead. Such partition-specific, cellular, or bead barcodes can be generated using a variety of methods, which methods result in the barcode conjugated to or incorporated into a solid or hydrogel support (e.g., a solid bead or particle or hydrogel bead or particle). In some cases, the partition-specific, cellular, or bead barcode is generated using a split and mix (also referred to as split and pool) synthetic scheme as described herein. A partition-specific barcode can be a cellular barcode and/or a bead barcode. Similarly, a cellular barcode can be a partition specific barcode and/or a bead barcode. Additionally, a bead barcode can be a cellular barcode and/or a partition-specific barcode.

In other cases, barcodes uniquely identify the molecule to which it is conjugated and are referred to as a unique molecular identifier (UMI). The number of nucleotides of the UMI, which can be continuous, or discontinuous, will depend on the number of UMI sequences required. In some embodiments, the number of UMIs available are many times (e.g., 2×, 10×, 100×, etc) higher than possible conjugation partners, thereby reducing the chance of rare duplicates being linked to different molecules. In some embodiments, pools of different UMIs are present in a partition and the composition of the pool acts as an identifiers for the partition, with some UMIs being in common with some other partitions but the total pool of UMIs being unique or substantially unique between partitions. UMI sequences can be generated for example as random sequences of a set length, and in some embodiments is identified by a flanking known sequence.

The length of the barcode sequence determines how many unique samples can be differentiated. For example, a 1 nucleotide barcode can differentiate 4, or fewer, different samples or molecules; a 4 nucleotide barcode can differentiate 4⁴ or 256 samples or less; a 6 nucleotide barcode can differentiate 4096 different samples or less; and an 8 nucleotide barcode can index 65,536 different samples or less. Additionally, barcodes can be attached to both strands either through barcoded primers for both first and second strand synthesis, through ligation, or in a tagmentation reaction.

Barcodes are typically synthesized and/or polymerized (e.g., amplified) using processes that are inherently inexact. Thus, barcodes that are meant to be uniform (e.g., a cellular, particle, or partition-specific barcode shared amongst all barcoded nucleic acid of a single partition, cell, or bead) can contain various N−1 deletions or other mutations from the canonical barcode sequence. Thus, barcodes that are referred to as “identical” or “substantially identical” copies refer to barcodes that differ due to one or more errors in, e.g., synthesis, polymerization, or purification errors, and thus contain various N−1 deletions or other mutations from the canonical barcode sequence. Moreover, the random conjugation of barcode nucleotides during synthesis using e.g., a split and pool approach and/or an equal mixture of nucleotide precursor molecules as described herein, can lead to low probability events in which a barcode is not absolutely unique (e.g., different from all other barcodes of a population or different from barcodes of a different partition, cell, or bead). However, such minor variations from theoretically ideal barcodes do not interfere with the high-throughput sequencing analysis methods, compositions, and kits described herein. Therefore, as used herein, the term “unique” in the context of a particle, cellular, partition-specific, or molecular barcode encompasses various inadvertent N−1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences). For example, 10,000 cells can be analyzed using a cellular barcode having 9 barcode nucleotides, representing 262,144 possible barcode sequences. The use of barcode technology is well known in the art, see for example Katsuyuki Shiroguchi, et al. Proc Natl Acad Sci USA., 2012 Jan. 24; 109(4):1347-52; and Smith, A M et al., Nucleic Acids Research Can 11, (2010). Further methods and compositions for using barcode technology include those described in U.S. 2016/0060621.

A “transposase” or “tagmentase” means an enzyme that is capable of forming a functional complex with a transposon end-containing composition and catalyzing insertion or transposition of the transposon end-containing composition into the double-stranded target DNA with which it is incubated in an in vitro transposition reaction.

The term “transposon end” means a double-stranded DNA that exhibits only the nucleotide sequences (the “transposon end sequences”) that are necessary to form the complex with the transposase that is functional in an in vitro transposition reaction. A transposon end forms a “complex” or a “synaptic complex” or a “transposome complex” or a “transposome composition with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a “transferred transposon end sequence” or “transferred strand” and a “non-transferred transposon end sequence,” or “non transferred strand” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that exhibits a “transferred transposon end sequence” as follows:

(SEQ ID NO: 1)
5′ AGATGTGTATAAGAGACAG 3′,

and a non-transferred strand that exhibits a “non-transferred transposon end sequence” as follows:

(SEQ ID NO: 2)
5′ CTGTCTCTTATACACATCT 3′.

The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

The term “solid support” refers to the surface of a bead, microtiter well or other surface that is useful for attaching a nucleic acid, such as an oligonucleotide or polynucleotide. The surface of the solid support can be treated to facilitate attachment of a nucleic acid, such as a single stranded nucleic acid.

The term “bead” refers to any solid support that can be in a partition, e.g., a small particle or other solid support. In some embodiments, the beads comprise polyacrylamide. For example, in some embodiments, the beads incorporate barcode oligonucleotides into the gel matrix through an acrydite chemical modification attached to each oligonucleotide. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

It will be understood that any range of numerical values disclosed herein can include the endpoints of the range, and any values or subranges in between the endpoints. For example, the range 1 to 10 includes the endpoints 1 and 10, and any value between 1 and 10. The values typically include one significant digit.

The term “sample” refers to a biological composition, such as a cell, comprising a target nucleic acid.

The term “deconvolution” refers to the assignment of 2 barcodes and the beads they were attached to as being from the same partition or originally occupying the same partition. Deconvolution can be determined by the detection of the two barcodes on a single nucleic acid fragment during sequencing.

The term “about” refers to the usual error range for the respective value that is known by a person of ordinary skill in the art for this technical field, for example, a range of ±10%, ±5%, or ±1% can encompass the recited value, even if the recited value is not modified by the term “about.”

All ranges described herein can include the end point values of the range, and any sub-range of values included between the endpoints of the range, where the values include the first significant digit. For example, a range of 1 to 10 includes a range from 2 to 9, 3 to 8, 4 to 7, 5 to 6, 1 to 5, 2 to 5, 2 to 10, 3 to 10, and so on.

### DETAILED DESCRIPTION OF THE INVENTION

Barcode bead overloading in partitions such as droplets increases droplet utilization, such that >90% of droplets contain at least one solid support (such as a bead) and are active during barcoding. However, to prevent the fractionation of the substrate (i.e., cell) data, and/or the over representation of the substrate (i.e., cell) when a partition has more than one solid support, solid supports need to be co-localized to single partitions. When this occurs, the data for each of the co-localized barcodes can be merged in silico to preserve data integrity.

In general, it can be desirable to use partition-specific barcodes to barcode all target nucleic acids in a partition with the same barcode such that the contents of different partitions can be later combined and nevertheless tracked back to their origins in different partitions in view of different reads having different partition-specific barcodes. Many multiple copies of partition-specific barcodes can be linked to a bead and delivered to a partition. However, methods of making partition-specific barcodes and delivering them into partitions (e.g., linked to beads) are often controlled by a Poisson distribution, meaning some partitions do not receive any partition-specific barcodes (e.g., any beads), some partitions receive only one partition-specific barcode (e.g., one bead) and some partitions receive two or more partition-specific barcodes (e.g., two or more beads). The latter creates issues in that if two different partition-specific barcodes occur in a partition, sequence reads having different partition-specific barcodes would be incorrectly interpreted as originating from different partitions. As described herein, methods are provided for determining when two partition-specific barcodes originate in the same partition and thus allows one to use data from many more partitions, indeed allowing for one to “overload” partitions with beads such that there are many fewer partitions that lack any beads.

The problems associated with more than one barcoded bead per partition can be solved by the methods and compositions described herein. The methods described herein involve initiating, in fixed and permeablized cells, transposase-mediated fragmentation and adaptor insertion to form “tagmented” fragments that comprise universal sequences as well as a unique molecule identifier (UMI) barcode in one or both adaptor sequences. Permeabilized cells carrying tagmented fragments are partitioned with, or added to partitions that contain, bead-linked oligonucleotides. The tagmented fragments in a single partitioned cell are subsequently gap-filled after cell lysis, whereas bead-linked oligonucleotides are subsequently linked to the gap-filled tagmented fragments. The resulting products are sequenced in bulk (following partition merging). Two different bead-linked oligonucleotides in the same partition can be identified by detecting the same UMI sequence linked in sequencing read to different bead-linked oligonucleotides, indicating they both likely originated in the same partition, because UMIs would be unlikely to occur in different partitions. In some embodiments, one can also gain confidence that the sequencing reads having different bead-linked oligonucleotide sequences and the same UMI originated in the same partition by further confirming the same fragment, i.e., having the same fragmented ends formed by the tagmentation, is present in both sequencing reads. Once it is established that two different bead-linked oligonucleotides occurred in the same partition, all sequencing reads containing either of the bead-linked oligonucleotide sequences can be assumed to be from the same partition, and thus for example all sequencing reads from both bead-linked oligonucleotides can be combined to represent sequencing results from the same partition.

The methods can involve either RNA/first strand cDNA hybrid molecules or DNA/cDNA hybrid molecules. RNA/first strand cDNA hybrid molecules can be generated by any way one forms first strand cDNAs, for example using a reverse transcriptase to form a first strand cDNA based on an RNA template. A DNA/cDNA hybrid molecule can be formed for example with a polymerase using the DNA strand as a template for the cDNA strand. Alternatively, any source of double-stranded DNA can be used. “Hybrid” in this context refers to a double-stranded nucleic acid duplex.

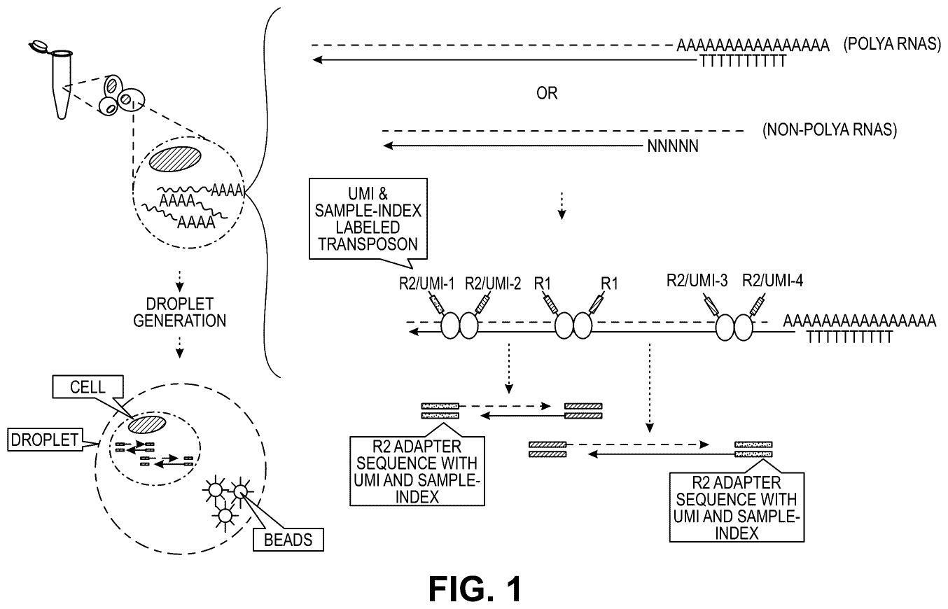

In the context of the methods described herein, the RNA/first strand cDNA hybrid molecules or DNA/cDNA hybrid molecules are provided in cells that are fixed and permeabilized. See, e.g., FIG. 1, item 1. Thus, the RNA/first strand cDNA hybrid molecules or DNA/cDNA hybrid molecules can be formed before, or in many embodiments, after the cells have been fixed and permeabilized to allowed to diffusion of reagents (e.g., a polymerase) into the cells, while substantially retaining nucleic acids within the fixed and permeabilized cells.

In some embodiments, the cells comprising target nucleic acids is a biological sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc. In some embodiments, the sample is a sample comprising cells. In some embodiments, the sample is a single-cell sample.

Formation of the RNA/first strand cDNA hybrid molecules by reverse transcription in the permeabilized cells can be achieved using polyT primers, random primers (e.g., random 6 mers for example) or gene-specific primer complementary for target RNA. See, e.g., FIG. 1, item 2. In some embodiments, the 3′ sequence is a polyT sequence of at least 5 contiguous thymines. In some embodiments, the 3′ sequence is a random sequence of at least 5 (e.g., at least 8, at least 10, at least 12, e.g., 6-30) contiguous nucleotides. In some embodiments, the 3′ sequence is a target gene-specific sequence of at least 5 contiguous nucleotides. Target RNA can be mRNA or non-polyA RNA. In some embodiments, the first strand cDNAs generated are full-length based on the corresponding RNA from which the cDNA was generated. A variety of reverse transcriptases can be used to form RNA/cDNA hybrids in the cells

Any methods of fixing and permeabilizing cells can be used. For example in some embodiments, the cells are formalin-fixed, paraffin-embedded (FFPE) samples. In fixed permeabilized cells, the reagents can be diffused into the cells for example to cause reverse transcription, cleaving, annealing and/or ligating to occur within the fixed cells. Exemplary fixative reagents can include the use of digitonin, or fixatives such as methanol (see, e.g., Alles, J., et al., BMC Biol 15, 44 (2017)), or paraformaldehyde. Permeabilization reagents can include, for example, Triton X-100. In some embodiments, the sample comprises target nucleic acids that are isolated from tissue or cells. In some embodiments, the cells will have intact chromatin such that some chromosomal regions are more accessible to the transposase than other chromosomal regions, allowing for ATACseq results to be generated. Exemplary conditions can include those for example, Nesterenko, et al., Proc. Nat. Acad. Sci. USA Vol. 118 No. 3 (2021). As noted below, in some embodiments, the fixation reagents are selected such that at least some of the cross-linking that occurs in fixation (e.g., protein-protein cross-linking) can be reversed at a later point, e.g., by heat less than 110° C. and or reversable reagents, e.g., such as reducing agents, or both.

Fragmentation and attachment of end adaptors on the RNA/first strand cDNA hybrid molecules or DNA/cDNA hybrid molecules can be achieved with a transposase. See, e.g., FIG. 1, item 3. The action of the transposase sometimes referred to as “tagmentation” and can involve introduction of different adapter sequences on different sides of a DNA breakage point caused by the transposase or the adapter sequences added can be identical. In either case, the one or two adapter sequences are common adapter sequences in that the adapter sequences are the same across a diversity of DNA fragments. Homoadapter-loaded tagmentases are tagmentases that contain adapters of only one sequence, which adapter is added to both ends of a tagmentase-induced breakpoint in the genomic DNA. Heteroadapter-loaded tagmentases are tagmentases that contain two different adapters, such that a different adapter sequence is added to the two DNA ends created by a tagmentase-induced breakpoint in the DNA. Adapter loaded tagmentases are further described, e.g., in U.S. Patent Publication Nos: 2010/0120098; 2012/0301925; and 2015/0291942 and U.S. Pat. Nos. 5,965,443; 6,437,109; 7,083,980; 9,005,935; and 9,238,671, the contents of each of which are hereby incorporated by reference in the entirety for all purposes. Tagmentation of RNA/DNA hybrids is described in, e.g., Bo LuLiting et al., eLife 9:e54919 (2020).

A tagmentase is an enzyme that is capable of forming a functional complex with a transposon end-containing composition and catalyzing insertion or transposition of the transposon end-containing composition into the double-stranded target DNA with which it is incubated in an in vitro transposition reaction. Exemplary transposases include but are not limited to modified Tn5 transposases that are hyperactive compared to wildtype Tn5, for example can have one or more mutations selected from E54K, M56A, or L372P. Wild-type Tn5 transposon is a composite transposon in which two near-identical insertion sequences (IS50L and IS50R) are flanking three antibiotic resistance genes (Reznikoff W S. Annu Rev Genet 42: 269-286 (2008)). Each IS50 contains two inverted 19-bp end sequences (ESs), an outside end (OE) and an inside end (IE). However, wild-type ESs have a relatively low activity and were replaced in vitro by hyperactive mosaic end (ME) sequences. A complex of the transposase with the 19-bp ME is thus all that is necessary for transposition to occur, provided that the intervening DNA is long enough to bring two of these sequences close together to form an active Tn5 transposase homodimer (Reznikoff W S., et al. Mol Microbiol 47: 1199-1206 (2003)). Transposition is a very infrequent event in vivo, and hyperactive mutants were historically derived by introducing three missense mutations in the 476 residues of the Tn5 protein (E54K, M56A, L372P), which is encoded by IS50R (Goryshin I Y, Reznikoff W S. 1998. J Biol Chem 273: 7367-7374 (1998)). Transposition works through a “cut-and-paste” mechanism, where the Tn5 excises itself from the donor DNA and inserts into a target sequence, creating a 9-bp duplication of the target (Schaller H. Cold Spring Harb Symp Quant Biol 43: 401-408 (1979); Reznikoff W S., Annu Rev Genet 42: 269-286 (2008)). In current commercial solutions (Nextera™ DNA kits, Illumina), free synthetic ME adapters are end-joined to the 5′-end of the target DNA by the transposase (tagmentase).

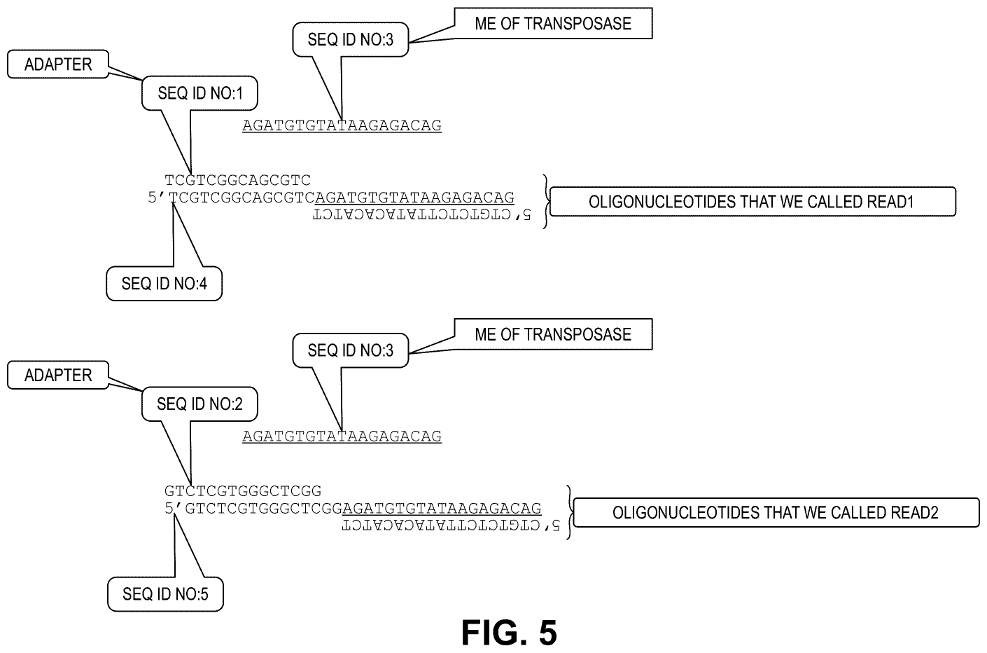

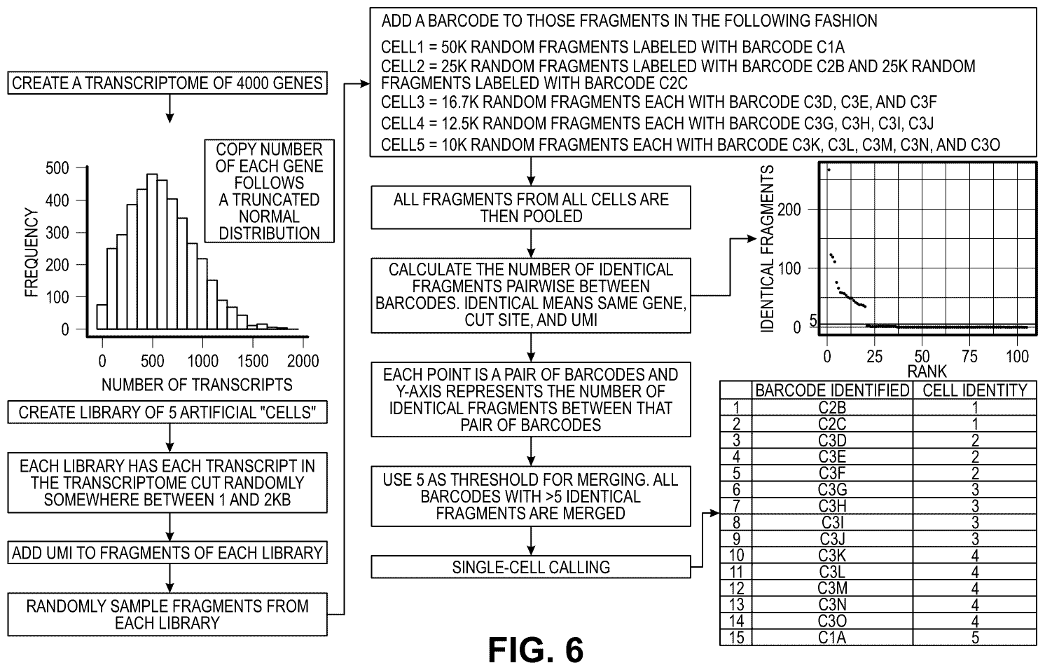

In some embodiments, the adapter(s) is at least 19 nucleotides in length, e.g., 19-100 nucleotides. In some embodiments, the adapters are double stranded with a 5′ end overhang, wherein the 5′ overhand sequence is different between heteroadapters, while the double stranded portion (typically 19 bp) is the same. In some embodiments, an adapter comprises TCGTCGGCAGCGTC (SEQ ID NO: 1) or GTCTCGTGGGCTCGG (SEQ ID NO:2). In some embodiments involving the heteroadapter-loaded tagmentase, the tagmentase is loaded with a first adapter comprising TCGTCGGCAGCGTC (SEQ ID NO:1) and a second adapter comprising GTCTCGTGGGCTCGG (SEQ ID NO:2). In some embodiments, the adapter comprises AGATGTGTATAAGAGACAG (SEQ ID NO:3) and the complement thereof (this is the mosaic end and this is the only specifically required cis active sequence for Tn5 transposition). In some embodiments, the adapter comprises TCGTCGGCAGCGTCAGATGTGTATAAGAGACAG (SEQ ID NO:4) with the complement for AGATGTGTATAAGAGACAG (SEQ ID NO:3) or GTCTCGTGGGCTCGGAGATGTGTATAAGAGACAG (SEQ ID NO: 5) with the complement for AGATGTGTATAAGAGACAG (SEQ ID NO:3). In some embodiments involving the heteroadapter-loaded tagmentase, the tagmentase is loaded with a first adapter comprising TCGTCGGCAGCGTCAGATGTGTATAAGAGACAG (SEQ ID NO:4) with the complement for AGATGTGTATAAGAGACAG (SEQ ID NO:3) and GTCTCGTGGGCTCGGAGATGTGTATAAGAGACAG (SEQ ID NO: 5) with the complement for AGATGTGTATAAGAGACAG (SEQ ID NO:3). See, e.g., FIG. 5.

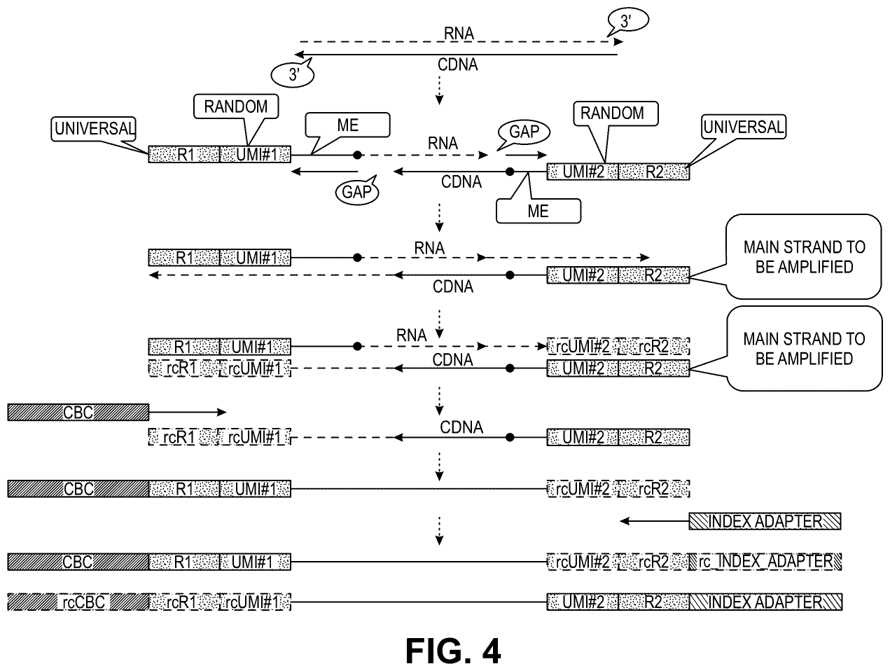

The transposase generates random breaks in the hybrid molecules and randomly inserts at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence and wherein the first adaptor oligonucleotide, the second adaptor oligonucleotide, or both further comprise a unique molecular identifier (UMI) sequence. See, e.g., FIG. 4. In some embodiments, the transposase is used loaded as a heteroadaptor such that two different adaptors can be attached to breaks in the target hybrid nucleic acid. Alternatively, two transposases can be used wherein a first transposase is loaded with one set of adaptor oligonucleotides (e.g., for ease of description the “first adaptor oligonucleotide”) and a second transposase is loaded with a second set of adaptor oligonucleotides, (the “second adaptor oligonucleotide”) thereby allowing, by random fragmentation by the two transposases, the formation of fragments that have the first adaptor oligonucleotide on a first end and the second adaptor on the second end. An “adaptor oligonucleotide” refers to an oligonucleotide that carries a universal sequences, wherein the universal sequences are common to end sequences between different fragments to which the oligonucleotide adaptor is attached and permit their use as PCR handle sequences, allowing one pair of universal primers to amplify different fragments that have the universal sequences.

The products of the transposase-based fragmentation are hybrid molecule fragments (“hybrid” in this context meaning double-stranded nucleic acids) comprising in a first strand: (i) a first 5′ end linked to a first adaptor oligonucleotide and (ii) a first 3′ end and in a second strand: (iii) a second 5′ end linked to a second adaptor oligonucleotide and (iv) a second 3′ end. Because at least one, and optionally both, the first adaptor oligonucleotide and the second adaptor oligonucleotides comprise a UMI sequence, at least some hybrid molecule fragments will be generated that include a UMI sequence. The first adaptor oligonucleotide and second adaptor oligonucleotide comprise a universal sequence and optionally a UMI sequence. The universal sequence can be any length as desired, and will generally be useful as PCR handle sequences, allowing for subsequent use of universal primers that hybridize to the universal sequences to amplify the hybrid molecule fragments having the appropriate adaptor sequences. In some embodiments, the universal sequence is 4-20 nucleotides long, e.g., 6-12 nucleotides long. The universal sequence in each first adaptor oligonucleotide will be identical; similarly the universal sequence in each second adaptor oligonucleotide will be identical. But in some embodiments, the universal sequences of the first adaptor oligonucleotide and the second adaptor oligonucleotide will be different.

The UMI sequence, in contrast, will ideally be different for every adaptor oligonucleotide. The number of nucleotides of the UMI sequence will depend on the number of fragments used. Ideally, the number of unique UMI sequences will be greater (e.g., 2×, 10×, 50, etc.) than the number of fragments. The UMI sequence can be contiguous or be formed of non-contiguous nucleotides. Exemplary UMI sequences are for example, 5-20 nucleotides long, e.g., 8-16 nucleotides long.

Following transposition (tagmentation), the permeabilized cells containing the transposases-treated nucleic acids having adaptor sequences at their ends can be added to partitions or partitioning can be performed in the presence of the permeabilized cells. Ideally most cells in the partitions are the only cell in the partition and this can be achieved for example according to Poisson distribution by allowing for a certain number of empty (no cell) partitions. Based on a Poisson distribution, one can calculate the probability of having single-cell partition depending on the ratio of cells and available partitions. In some embodiments, a ratio of 1 cell to 10 partitions is used, e.g., 0.5-2 cells per 10 partitions.

In some embodiments, in the partitions, the cell fixation can be at least partially reversed, allowing for improved accessibility of tagmented nucleic acids for priming, and/or the cells are lysed, thereby enhancing capture efficiency of the barcoding. Preferably, reversal of crosslinking of a cell occurs in partitions so that the cellular content is held in the partition for barcoding. Reversal of fixation can occur before, during or after the gap-filling. Reversal of fixation can involve, but is not limited to, applying heat (e.g., 95-98° C.), applying proteases to degrade cross-linked proteins, addition of reducing agents, or a combination thereof. See, e.g., Namimatsu et al., J Histochem Cytochem. 2005 January; 53(1):3-11. Cell lysis can comprise for example contacting the cells with a detergent compatible with the partitions, heat or other condition to cause lysis.

In some embodiments, the tagmentase is stripped from the nucleic acids before proceeding. For example, in some embodiments, the tagmented nucleic acids in permeabilized cells are not gap-filled until transposase proteins bound to the nucleic acids are stripped off. In some embodiments, the transposase proteins can be stripped from the nucleic acid using, for example, SDS or heating (e.g., to about 80-82° C.), thereby exposing the gap/3′ end for polymerization.

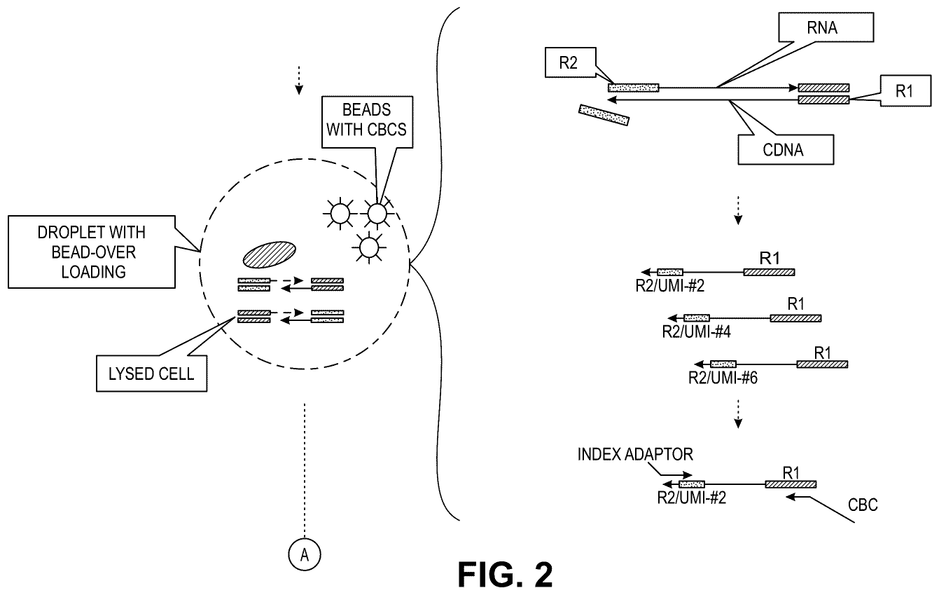

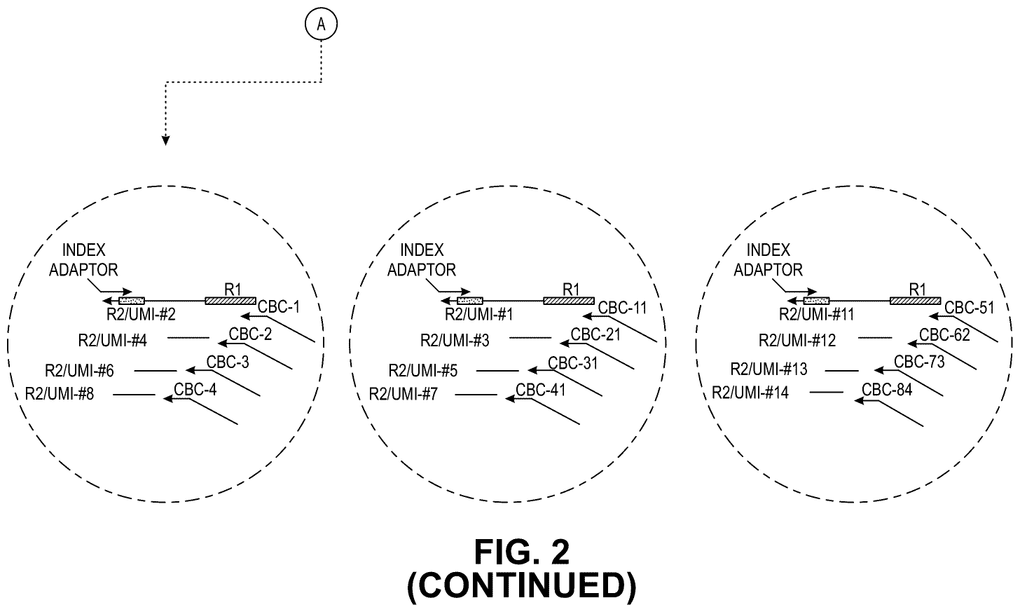

A subsequent gap-filling step can be performed on the hybrid molecule fragments formed from the tagmentation. See, e.g., FIG. 2, item 5. For example, a polymerase, e.g., a DNA polymerase can be used to fill in any gaps, for example at the ends of the hybrid molecule fragments having adaptor sequences at their ends. In general, in embodiments involving RNA/cDNA hybrids, extension from the RNA is less efficient than extension of the cDNA (using RNA as a template). As a result, the cDNA strand is the main strand being amplified in the downstream reaction, as depicted for example with the bold dot line of FIG. 4.

Methods and compositions for partitioning are described, for example, in published patent applications WO 2010/036,352, US 2010/0173,394, US 2011/0092,373, and US 2011/0092,376, the contents of each of which are incorporated herein by reference in the entirety. The plurality of mixture partitions can be in a plurality of emulsion droplets, or a plurality of microwells, etc.

In some embodiments, the primers and other reagents can be partitioned into a plurality of mixture partitions, and then linked DNA segments can be introduced into the plurality of mixture partitions. Methods and compositions for delivering reagents to one or more mixture partitions include microfluidic methods as known in the art; droplet or microcapsule merging, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

As described herein, the mixture partitions can be picowells, nanowells, or microwells. The mixture partitions can be pico-, nano-, or micro-reaction chambers, such as pico, nano, or microcapsules. The mixture partitions can be pico-, nano-, or micro-channels. The mixture partitions can be droplets, e.g., emulsion droplets.

In some embodiments, the partitions are droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, %, 21%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. In some cases, such stability or minimal coalescence is maintained for up to 4, 6, 8, 10, 12, 24, or 48 hours or more (e.g., at room temperature, or at about 0, 2, 4, 6, 8, 10, or 12° C.). In some embodiments, the droplet is formed by flowing an oil phase through an aqueous sample or reagents.

The oil phase can comprise a fluorinated base oil which can additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules can behave as bioreactors able to retain their contents through an incubation period. The conversion to microcapsule form can occur upon heating. For example, such conversion can occur at a temperature of greater than about 40°, 50°, 60°, 70°, 80°, 90°, or 95° C. During the heating process, a fluid or mineral oil overlay can be used to prevent evaporation. Excess continuous phase oil can be removed prior to heating, or left in place. The microcapsules can be resistant to coalescence and/or flocculation across a wide range of thermal and mechanical processing.

Following conversion of droplets into microcapsules, the microcapsules can be stored at about −70°, −20°, 0°, 3°, 4°, 5°, 6°, 7°, 8°, 9°, 10°, 15°, 20°, 25°, 30°, 35°, or 40° C. In some embodiments, these capsules are useful for storage or transport of partition mixtures. For example, samples can be collected at one location, partitioned into droplets containing enzymes, buffers, and/or primers or other probes, optionally one or more polymerization reactions can be performed, the partitions can then be heated to perform microencapsulation, and the microcapsules can be stored or transported for further analysis.

In some embodiments, the sample is partitioned into, or into at least, 500 partitions, 1000 partitions, 2000 partitions, 3000 partitions, 4000 partitions, 5000 partitions, 6000 partitions, 7000 partitions, 8000 partitions, 10,000 partitions, 15,000 partitions, 20,000 partitions, 30,000 partitions, 40,000 partitions, 50,000 partitions, 60,000 partitions, 70,000 partitions, 80,000 partitions, 90,000 partitions, 100,000 partitions, 200,000 partitions, 300,000 partitions, 400,000 partitions, 500,000 partitions, 600,000 partitions, 700,000 partitions, 800,000 partitions, 900,000 partitions, 1,000,000 partitions, 2,000,000 partitions, 3,000,000 partitions, 4,000,000 partitions, 5,000,000 partitions, 10,000,000 partitions, 20,000,000 partitions, 30,000,000 partitions, 40,000,000 partitions, 50,000,000 partitions, 60,000,000 partitions, 70,000,000 partitions, 80,000,000 partitions, 90,000,000 partitions, 100,000,000 partitions, 150,000,000 partitions, or 200,000,000 partitions.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, the standard deviation of droplet volume can be less than about 1 picoliter, 5 picoliters, 10 picoliters, 100 picoliters, 1 nL, or less than about 10 nL. In some cases, the standard deviation of droplet volume can be less than about 10-25% of the average droplet volume. In some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

Also in the partitions, resulting from the partitioning, or by addition to formed partitions, is one or more bead, where the beads are linked to copies of an oligonucleotide that will allow for bead-specific (and to some extent partition-specific) barcoding of the fragments. See, e.g., FIG. 2, item 4. The bead can be attached to multiple copies of the same oligonucleotide, for example, at least about 10, 50, 100, 500, 1000, 5000, 10,000, 50,000, 100,000, 500,000, 1,000,000, 5,000,000, 10,000,000, 10⁸, 10⁹, 10¹⁰ or more copies of the same or substantially identical oligonucleotide can be attached to one (e.g., the same) bead.

Due to Poisson distribution of beads into partitions, at least some partitions contain at least two different of the beads. See, e.g., FIG. 1 or FIG. 2, item 4. During partitioning, more than one barcode bead can be encapsulated in single partitions due to Poisson distributions statistics. Indeed, because the present methods allow one to detect and deconvolute the presence of multiple bead specific barcodes in a single partition, the number of beads delivered to partitions in the methods described herein can be such that a large number of partitions have more than one bead. This is advantageous because a number of partitions can be left empty if a method can only accurately using partitions with single beads, whereas if one can deconvolute data from multiple beads in a partition, the number of partitions containing at least one bead can be increased. Thus, in some embodiments, one can select conditions such that at least 10%, 20%, 30%, 40%, 50% or more partitions contain at least one or alternatively, more than one bead.

Each oligonucleotide can be linked at its 5′ end or elsewhere on the oligonucleotide to the bead and in some embodiments can include a cleavable moiety to remove the oligonucleotides from the bead, e.g., before the oligonucleotides are used for primer extension. In some embodiments, the cleavable linker comprises a uridine incorporated site in a portion of a nucleotide sequence. A uridine incorporated site can be cleaved, for example, using a uracil glycosylase enzyme (e.g., a uracil N-glycosylase enzyme or uracil DNA glycosylase (UDG) enzyme). In some embodiments, the cleavable linker comprises a photocleavable nucleotide. Photocleavable nucleotides include, for example, photocleavable fluorescent nucleotides and photocleavable biotinylated nucleotides. See, e.g., Li et al., PNAS, 2003, 100:414-419; Luo et al., Methods Enzymol, 2014, 549:115-131. In some cases, the oligonucleotides are attached to bead through a disulfide linkage (e.g., through a disulfide bond between a sulfide of the solid support and a sulfide covalently attached to the 5′ or 3′ end, or an intervening nucleic acid, of the oligonucleotide). In such cases, the oligonucleotide can be cleaved from the solid support by contacting the solid support with a reducing agent such as a thiol or phosphine reagent, including but not limited to a beta mercaptoethanol, dithiothreitol (DTT), or tris(2-carboxyethyl)phosphine (TCEP).

The oligonucleotides from the bead will include, for example, a bead-specific barcode such that the bead-specific barcode sequence on a first oligonucleotide can be used to distinguish it from a bead-specific barcode from a second oligonucleotide on a different bead. The 3′ end of the oligonucleotides will comprise the reverse complement of one of the universal sequences from the adaptor sequences added to the fragments such that the oligonucleotides from the beads can be used as primers in a primer extension (e.g., PCR) reaction using one strand of the gap-filled hybrid molecule fragments having adaptor sequences at their ends as a template. See, e.g., FIG. 2, item 6, bead-specific barcodes are indicated as “CBC”). This can be achieved for example in embodiments in which a DNA polymerase is included in the partitions along with reagents such as salts for conditions allowing for polymerase activity. Thus, resulting templates become linked to a bead-specific barcode from the oligonucleotides within the partitions. Optionally, a reverse primer can be included in the partitions. The reverse primer can hybridize to the second universal sequence, or reverse complement thereof, on the opposite end of the gap-filled hybrid molecule fragments. See, e.g., FIG. 2, item 6, “index adaptor”, and also FIG. 4 in the context of RNA/cDNA.

Following primer extension with the bead-specific barcode oligonucleotide, the gap-filled hybrid molecule fragments will also include a bead-specific barcode. See, e.g., FIG. 2, item 5. Because some partitions will contain two or more beads, and thus oligonucleotides containing different bead-specific barcodes, different extension products (e.g., amplicons) will have different bead-specific barcodes. See, e.g., FIG. 2, item 7. The amplicons will have, for example, the bead-specific barcode, a first universal end sequence (or complement thereof), cDNA fragments, the second universal end sequence (or complement thereof) and the UMI sequence (or complement thereof).

Once the amplicons descried above are formed, the contents of the partitions can be combined to form a bulk solution comprising the contents of a plurality of the partitions. The amplicons in the resulting bulk solution can be nucleotide sequenced. Any method of nucleotide sequencing can be used as desired to form sequencing reads that include the bead-specific barcode, a first universal end sequence (or complement thereof), cDNA fragments, the second universal end sequence (or complement thereof) and the UMI sequence (or complement thereof). Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7:287-296; U.S. Pat. Nos. 6,210,891; and 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55. 641-658, 2009; MacLean et al., Nature Rev. Microbial., 7:287-296; U.S. Pat. Nos. 6,833,246; 7,115,400; and 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbial., 7:287-296; U.S. Pat. Nos. 5,912,148; and 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (See, e.g., Astier et al., J. Am. Chem. Soc. 2006 Feb. 8; 128(5)1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences is employed (Voelkerding et al., Clinical Chem., 55. 641-658, 2009; MacLean et al., Nature Rev. Microbial, 7:287-296; U.S. Pat. Nos. 7,169,560; 7,282,337; 7,482,120; 7,501,245; 6,818,395; 6,911,345; and 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (See, e.g., Science 327(5970): 1190 (2010); U.S. Pat. Appl. Pub. Nos. 2009/0026082; 2009/0127589; 2010/0301398; 2010/0197507; 2010/0188073; and 2010/0137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is ^{˜}99.6% for 50 base reads, with ^{˜}100 Mb generated per run. The read-length is 100 base pairs. The accuracy for homopolymer repeats of 5 repeats in length is ^{˜}98%. The benefits of ion semiconductor sequencing are rapid sequencing speed and low upfront and operating costs.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 2009/0035777, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; and U.S. patent application Ser. Nos. 11/671,956; and 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55. 641-658, 2009; MacLean et al., Nature Rev. Microbiol., 7:287-296; U.S. Pat. Nos. 7,170,050; 7,302,146; 7,313,308; and 7,476,503; all of which are herein incorporated by reference) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10⁻²¹ L). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the invention are described in, for example, U.S. Pat. Nos. 7,405,281; 7,315,019; 7,313,308; 7,302,146; and 7,170,050; and U.S. Pat. Pub. Nos. 2008/0212960; 2008/0206764; 2008/0199932; 2008/0199874; 2008/0176769; 2008/0176316; 2008/0176241; 2008/0165346; 2008/0160531; 2008/0157005; 2008/0153100; 2008/0153095; 2008/0152281; 2008/0152280; 2008/0145278; 2008/0128627; 2008/0108082; 2008/0095488; 2008/0080059; 2008/0050747; 2008/0032301; 2008/0030628; 2008/0009007; 2007/0238679; 2007/0231804; 2007/0206187; 2007/0196846; 2007/0188750; 2007/0161017; 2007/0141598; 2007/0134128; 2007/0128133; 2007/0077564; 2007/0072196; and 2007/0036511; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

Sequencing reads can be assigned to a specific partition, and thus identified as from the cell within the partition based on the bead-specific barcode linked to a sequence read. However, because multiple beads, and thus bead-specific barcodes, can be in one partition, the methods described herein can be used to determine when for example two or more beads originate in a specific partition and allows for application of sequencing reads having any of the relevant two or more bead-specific barcodes. Sequencing reads having different bead-specific barcodes, but being from the same partition, can be identified by the presence of the same UMI in different sequence reads having different bead-specific barcodes. Because UMIs are unique, or nearly unique, the occurrence of the same UMI sequence linked to different bead-specific barcodes in different sequencing reads indicates that the different sequencing reads originated from the same partition and thus the different sequencing reads can be compiled with other sequencing reads from the same partition. Note that once it has been determined that two bead-specific barcodes occurred in the same partition, all sequencing reads having either bead-specific barcode can be compiled in the same partition, regardless of whether all of the sequencing reads have the same UMI. This is because only a portion, likely a small portion, of events primed by the two different bead-specific barcode oligonucleotides will prime extension from the same fragment having the same UMI.

Optionally, one can also use the exact fragment as a further way to determine that two different sequencing reads having different bead-specific barcodes and the same UMI are in fact from the same partition. In some embodiments, UMI sequences can be generated randomly with a higher number of UMI options than copies the UMI is linked to, resulting in a low, but possible, chance that two different fragments will be linked to the same UMI. In other embodiments, pools of UMI sequences can be used such that a partition is identified by the presence of absence of one or more UMI. Nevertheless, as the number of UMI sequences is reduced for example to conserve reagents, the chance that, via random occurrence, the same UMI unique or pool of UMIs, will be linked to two different bead-specific barcodes by chance increases (albeit still remaining relatively low). To distinguish from these rare events, one can also consider the cDNA fragment itself. If two sequencing reads with different bead-specific barcodes have the same UMI (or pool of UMIs)—and also the fragment is the same (same endpoints, optionally identical sequence)—then the sequencing reads can be considered from the same partition, whereas if the fragment ends or sequence differ, then the sequencing reads can be discarded or at least the bead-specific barcodes cannot be considered as originating from the same partition.

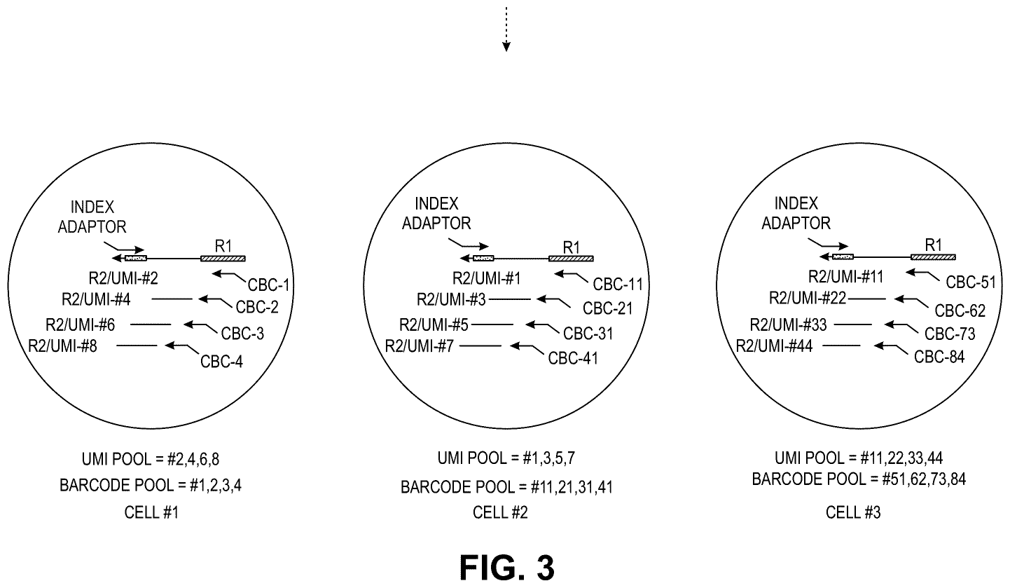

Accordingly in some embodiments, the methods described herein comprise sorting sequencing reads from different partitions wherein (i) the amplicon comprising the first bead-specific barcode and the first UMI sequence and (ii) the amplicon comprising the second bead-specific barcode and the first UMI sequence are from the same partition. See, e.g., FIG. 3 for an example of deconvolution.

In another aspect, compositions are provided comprising partitions comprising hybrid molecule fragments as described herein (i.e., double-stranded nucleic acids) comprising in a first strand: (i) a first 5′ end linked to a first adaptor oligonucleotide and (ii) a first 3′ end and in a second strand: (iii) a second 5′ end linked to a second adaptor oligonucleotide and (iv) a second 3′ end, In other aspects, compositions are provided comprising partitions comprising the gap-filled hybrid molecule fragments described herein. In other aspects, compositions are provided comprising partitions comprising permeabilized cells containing the gap-filled hybrid molecule fragments having adaptor sequences at their ends as described herein. In other aspects, compositions are provided comprising partitions comprising extension products (e.g., amplicons) comprising a bead-specific barcode are provided. In some embodiments, at least some of the partitions comprise extension products (e.g., amplicons) having different bead-specific barcodes. Any of the partitions described above can include other reagents described herein, e.g., a polymerase, one or more bead, or other reagents.

Also provided are reaction mixtures described herein. For example, a bulk solution comprising the extension products (e.g., amplicons) having different bead-specific barcodes as described herein.

### EXAMPLE

### Example 1

To simulate the process of barcode-merging for deconvolution of multi-barcodes partition in silico, a computer-based experimental process was designed to describe and verify the invention. This involves single-cell transcriptome creation, random fragmentation and UMI tagging, barcode sequence attachment, as well as barcode merging and single-cell containing partition calling.

A single-cell transcriptome was created from 4000 hypothetical genes. The copy number of each gene follows a truncated normal distribution of minimum 20 copies and maximum 4000 copies with a standard deviation of 400 copies. Five libraries representing 5 individual single-cell containing partitions were created using this transcriptome, where each library has each transcript in the transcriptome cut randomly somewhere between 1-2 kb and then added with UMI. After random sampling of fragments from each library, different fashions of barcode combination were added to those fragments to mimic single or multiple barcodes scenario in a partition as illustrated in process flow diagram. All fragments were then pooled before single-cell partition deconvolution step.

Single-cell containing partitions were identified by merging barcodes that most likely co-exist during barcoding step. In this process, the number of identical fragments, where identical is defined as same cut site and UMI, pairwise between barcodes were calculated and graphed as an Identical Fragment against Rank plot, of which each point is a pair of barcodes and y-axis represents the number of identical fragments (i.e. same gene, cut-site and UMI) between that pair of barcodes. Based on the inflection point, a threshold of 5 was determined so that barcodes with more than 5 identical fragments were grouped into corresponding bin that represents a cell-containing partition which was shown as Cell Identity in the table.

### Example 2

In order to verify the process of barcode-merging for deconvolution of multi-barcode partitions in a single-cell RNAseq experiment, we constructed transposome complexes loaded with UMI-indexed adapters as described herein. Following cell fixation and permeation, the transcriptome of individual cells was converted to cDNA in-situ and kept in the format of cDNA:RNA hybrid before being tagmented by using the UMI-index loaded transposome. Tagmented single-cells from human and mouse species were pooled in equal molar ratio and then partitioned together with barcode-beads in a droplet-emulsion based microfluidic device for single-cell barcoding. Each partition carries various number of barcode-beads, and either none or one tagmented cell following POISSON distribution probability. During barcoding, tagmented cDNA fragments were tagged with barcodes present in the partition. After the barcoding steps, barcoded-fragments were recovered, pooled and then subjected to sequencing analysis in bulk. Following the same approach described in the in-silico simulation test, single-cell-containing partitions were bioinformatically identified by merging barcodes that most likely co-exist during the barcoding step.

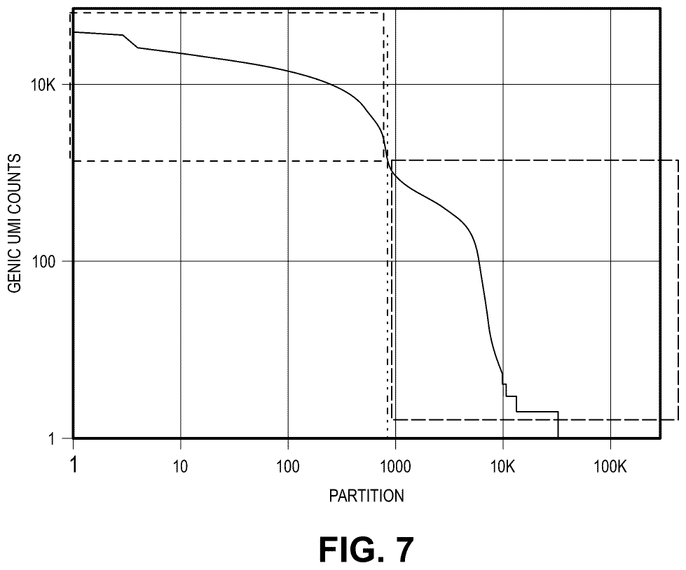

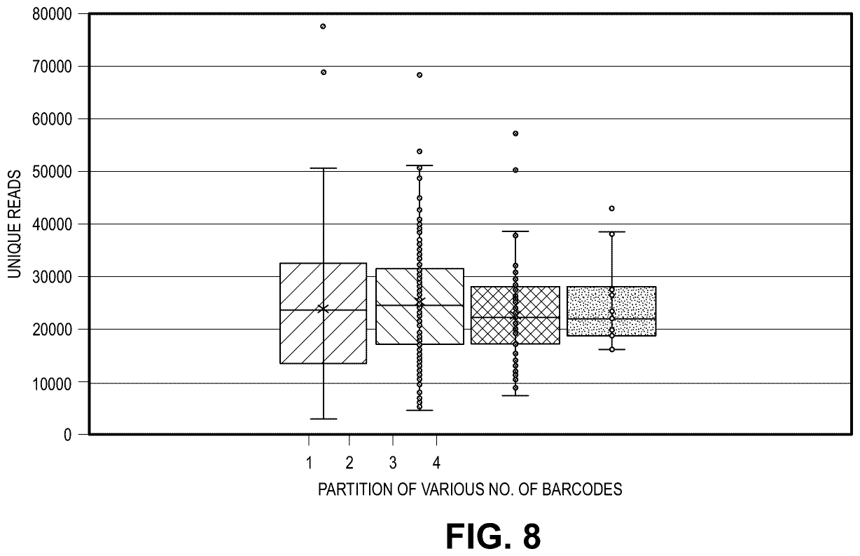

Success of barcode-merging for deconvolution of multi-barcode partition was reflected by having single-cell detection from the RNAseq experiment. FIG. 7 shows a ranking plot of arranging the detected partitions (after barcode-merging) with descending order of read counts. The top left dash box represents single-cell containing partitions. The bottom right dash box represents background and empty partitions. The vertical line is the threshold cutoff of determining the total number of single-cells. The evidence of barcode-merging for deconvolution of multi-barcode partition was further confirmed by plotting the total number of unique reads per detected partitions that have various number of detected barcodes (FIG. 8). The success of barcode-merging was reflected by having similar number of unique reads per partition regardless the number of barcode beads co-exist in that partition.

Although the foregoing disclosure has been described in some detail by way of illustration and example for purposes of clarity of understanding, one of skill in the art will appreciate that certain changes and modifications may be practiced within the scope of the appended claims. In addition, each reference provided herein, including patents, patent applications, non-patent literature, and Genbank accession numbers, is incorporated by reference in its entirety to the same extent as if each reference was individually incorporated by reference. Where a conflict exists between the instant application and a reference provided herein, the instant application shall dominate.

What is claimed is:

1. A method of sorting sequencing reads by partition of origin, the method comprising,
providing RNA/cDNA or DNA/cDNA hybrid molecules in fixed and permeabilized cells, wherein the fixed and permeabilized cells comprise cross-linked molecules;
generating random breaks in the hybrid molecules and randomly inserting at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence and wherein the first adaptor oligonucleotide, the second adaptor oligonucleotide, or both further comprise a unique molecular identifier (UMI) sequence;
partitioning the cells into partitions with one or more bead, wherein each bead is linked to multiple copies of a bead-specific barcoding oligonucleotide having an identical 3′ end comprising either the first universal sequence or the second universal sequence, wherein bead-specific barcoding oligonucleotides linked to different beads can be identified by a unique bead-specific barcode in the bead-specific barcoding oligonucleotide and wherein at least some partitions contain at least two different of the beads;
reversing at least some of the cross-linking in the cross-linked molecules in the cells;
before, during or after the reversing, extending with a polymerase (i) the first 3′ end using the second adaptor oligonucleotide as a template such that the first 3′ end is linked to a reverse complement of the second universal sequence and (ii) the second 3′ end using the first adaptor oligonucleotide as a template such that the second 3′ end is linked to a reverse complement of the first universal sequence, thereby forming gap-filled hybrid molecule fragments;
amplifying in the partitions the gap-filled hybrid molecule fragments by annealing and extending the bead-specific barcoding oligonucleotide to the reverse complements of the first universal sequence or the reverse complements of the second universal sequence on the cDNA fragments to generate amplicons comprising:
the bead-specific barcoding oligonucleotide, cDNA fragments, the second universal end sequence and the UMI sequence,
under conditions in which if a first bead and a second bead are present in a partition, bead-specific barcoding oligonucleotides from the first and second beads each separately are extended using the same cDNA hybrid molecule fragment as a template to form (i) an amplicon comprising a first bead-specific barcode and a first UMI sequence and (ii) an amplicon comprising a second bead-specific barcode and the first UMI sequence;
nucleotide sequencing amplicons from the amplifying to generate sequencing reads; and
sorting sequencing reads from different partitions wherein (i) the amplicon comprising the first bead-specific barcode and the first UMI sequence and (ii) the amplicon comprising the second bead-specific barcode and the first UMI sequence, and (iii) optionally a same fragment break point, are from the same partition.

2. The method of claim 1, wherein the hybrid molecules are RNA/cDNA hybrid molecules and the cDNA is a first strand cDNA.

3. The method of claim 2, wherein the RNA/first strand cDNA hybrid molecules are formed by reverse transcribing RNA from the cell with a polyA, random or gene-specific reverse transcription primer.

4. The method of claim 1, wherein the hybrid molecules are DNA/cDNA hybrid molecules.

5. The method of claim 4, wherein the DNA/first strand cDNA hybrid molecules are formed by polymerase chain reaction or primer extension.

6. The method of claim 1, wherein the first adaptor oligonucleotide comprises a UMI sequence.

7. The method of claim 1, wherein the second adaptor oligonucleotide comprises a UMI sequence.

8. The method of claim 1, wherein the first adaptor oligonucleotide and the second adaptor oligonucleotide comprises a UMI sequence.

9. The method of claim 1, wherein the first adaptor oligonucleotide, the second adaptor oligonucleotide, or both further comprises a sample barcode sequence.

10. The method of claim 1, wherein the generating comprises contacting the RNA/cDNA hybrid molecules with a transposase that introduces the adaptor oligonucleotides into the RNA/cDNA hybrid molecules.

11. The method of claim 1, wherein the bead-specific barcoding oligonucleotide comprises a 3′ end comprising the first universal sequence and the amplifying comprises annealing and extending the bead-specific barcoding oligonucleotide to the reverse complements of the first universal sequence.

12. The method of claim 11, wherein the amplifying further comprises extending a reverse primer having a 3′ end comprising a reverse complement of the second universal sequence using the amplicons as templates.

13. The method of claim 1, wherein the bead-specific barcoding oligonucleotide comprises a 3′ end comprising the second universal sequence and the amplifying comprises annealing and extending the bead-specific barcoding oligonucleotide to the reverse complements of the second universal sequence.

14. The method of claim 13, wherein the amplifying further comprises extending a reverse primer having a 3′ end comprising the first universal sequence using the amplicons as templates.

15. The method of claim 1, wherein the partitions are microwells or droplets in an emulsion.

16. The method of claim 1, wherein the cells are mammalian cells.

17. The method of claim 1, wherein the cells as prokaryotic cells.

18. The method of claim 1, wherein the cells are eukaryotic cells.

19. A plurality of partitions, at least some partitions comprising,
fixed and permeabilized cells comprising cross-linked molecules and containing
gap-filled hybrid molecule fragments formed by:
generating random breaks in RNA/cDNA or DNA/cDNA hybrid molecules and randomly inserting at the breaks first adaptor oligonucleotides or second adaptor oligonucleotides, thereby forming hybrid molecule fragments comprising (i) a first 5′ end linked to a first adaptor oligonucleotide and (ii) a first 3′ end and (iii) a second 5′ end linked to a second adaptor oligonucleotide and (iv) a second 3′ end, wherein the first adaptor oligonucleotide comprises a first universal sequence and the second adaptor oligonucleotide comprises a second universal sequence and wherein the first adaptor oligonucleotide, the second adaptor oligonucleotide, or both further comprise a unique molecular identifier (UMI) sequence;
partitioning the cells into partitions with one or more bead, wherein each bead is linked to multiple copies of a bead-specific barcoding oligonucleotide having an identical 3′ end comprising either the first universal sequence or the second universal sequence, wherein bead-specific barcoding oligonucleotides linked to different beads can be identified by a unique bead-specific barcode in the bead-specific barcoding oligonucleotide and wherein at least some partitions contain at least two different of the beads; and
extending with a polymerase (i) the first 3′ end using the second adaptor oligonucleotide as a template such that the first 3′ end comprises a reverse complement of the second universal sequence and (ii) the second 3′ end using the first adaptor oligonucleotide as a template such that the second 3′ end comprises a reverse complement of the first universal sequence, thereby forming gap-filled hybrid molecule fragments;
wherein the at least some partitions further comprise the one or more bead.

20. The plurality of partitions of claim 19, wherein the partitions are droplets in an emulsion or microwells.
